(19) United States
(12) Patent Application Publication
(10) Pub. No.: US 2026/0242736 A1
(43) Pub. Date: Aug. 20, 2026

(54) NOVEL PROCESS

(71) Applicant: NANODENT S.R.L., Ancona (IT)

(72) Inventors: Margherita De Angelis, Ancona (IT); Michela Cok, Ancona (IT)

(73) Assignee: NANODENT S.R.L., Ancona (IT)

(21) Appl. No.: 19/469,577

(22) PCT Filed: Mar. 27, 2024
(86) PCT No.: PCT/IB2024/052954

§ 371 Date: Sep. 26, 2025

(30) Foreign Application Priority Data
Mar. 28, 2023 (IT) 102023000005946

(51) Int. Cl. C12N 5/077; A61C 13/00; B33Y 70/10; B33Y 80/00

(52) U.S. Cl. CPC C12N 5/0654; A61C 13/0019; B33Y 70/10; B33Y 80/00; C12N 2506/1307; C12N 2533/18; C12N 2533/54; C12N 2533/74

(57) ABSTRACT

The present invention relates to a process for obtaining a dental prosthesis comprising a crown and tooth root. Said process comprises at least one step of directing undifferentiated cells or keratinocytes and at least one three-dimensional printing step, preferably 3D bioprinting of the dental prosthesis. Furthermore, the invention regards the dental prosthesis obtained With the process and a bioink suitable for three-dimensional printing (3D), preferably suitable for 3D bioprinting.

### FIELD OF THE INVENTION

The present invention relates to a process for obtaining a dental prosthesis comprising a crown and tooth root.

### STATE OF THE ART

Human teeth perform numerous functions such as, for example, chewing food and enabling a correct pronunciation of sounds. Furthermore, teeth play an important role in a person's aesthetic appearance. In fact, white, healthy, properly aligned teeth are an ideal of beauty and appear as an aesthetic sign of youth and success.

Although various preventive measures, such as frequent use of a toothbrush and dental floss, or the intake of fluoridated or iodinated water, are widely accepted and utilised, a large majority of people find themselves soon or later having to deal with tooth fillings, restoration implants and/or prostheses.

The main objective of dentistry is to prevent tooth loss or postpone it as long as possible. Another objective is certainly to provide comfortable prostheses with a wide range of application/indications and a long life.

In general, the number of available restorative and prosthetic options is limited: recourse is had to fillings, inlays, and crowns, if the root and the periodontal structure incorporating it are healthy and sufficient as a support for these partial prostheses. Traditionally, if the original tooth can no longer be utilised, the use of bridges or non-customised osseointegrated implants is indicated. In this context, various negative aspects must be put up with. In order to provide the support structure for a bridge, the adjacent teeth are shaved, and the heathy enamel is partly destroyed. Osseointegrated implants are drastically invasive and the gum-implant interface is often the cause of chronic local infections. Furthermore, all the restorative and prosthetic options mentioned have a limited average life. removable prostheses are certainly the definitive prosthetic option.

When a tooth is partially damaged, either because of cavities or mechanical impact, in most cases the missing part should be replaced. As long as the tooth has a sufficient structural strength to support a prosthesis, for example an inlay or a crown, this is the preferred solution. However, if the loss of tooth substance is severe, this solution might not be applicable. In these cases, a bridge can be applied, putting up with the negative consequences mentioned above. Another option is to replace the tooth with an implant.

There exist many methods or options to replace missing teeth. Premade or preformed osseointegrated dental implants are one of the options. Osseointegration means direct contact of the implant surface with the bone, without a fibrous connective tissue interface (natural teeth are not typically in direct contact with the bone but are rather connected to the bone by ligaments). The use of such dental implants includes a broad variety of implant designs and materials, the use of implants in different positions in the mouth and the use of a variety of surgical protocols.

At present, implants are generally made with titanium (recently also zirconia) elements which are cylindrical and/or cylindrical-conical in shape, threaded and provided with other accessory retainers, and covered with structured surfaces subjected to special treatments to increase the volume and osseointegration thereof; the abutment (which in some cases can also be an integral part of the screw) is fixed onto the implant and supports the crown that replicates the natural tooth.

The clinical protocols for “traditional” implants are usually rather long and complex (they generally involve about ten sittings) and, consequently, they are costly; recently, immediate loading implants have also been applied, but only in the rare cases in which all the appropriate clinical conditions are met—and in any case with sub-optimal results.

Furthermore, there also exist monoblock implants, made of zirconia; said material is not of a biological nature and the monoblock is limited to the screw and abutment, the crown in any case being a separate component. As regards bone grafts, bone regeneration techniques are by now widespread; they consist in the increase of bone by transplantation of the patient's own bone, or by using biocompatible external bone.

Besides the advantages of the prostheses presently on the market, in the literature there are data on the environmental impact of the different types of materials used in the dental field. The materials used include yttria-stabilised tetragonal zirconia polycrystal (Y-TZP), pure titanium (cp-Ti) and titanium and alpha-beta titanium alloy (Ti6Al4V). Considering, in fact, the ecological footprint of these materials, it is estimated that the production of cp-Ti and the Ti6Al4V alloy results in the emission of 39-46 kgCO₂/kg, whilst the production of zirconia results in the emission of 4.83 kgCO₂/kg. Furthermore, one should also consider the total energy expended in the processing of the aforesaid materials, which ranges from 89.5 MJ/kg for zirconia to 685.5 MJ/kg for the Ti6Al4V alloy.

Therefore, there is a strongly felt need to obtain biological dental prostheses using techniques capable of drastically reducing CO₂ emissions and the necessary energy consumption, reducing material waste, optimising the use of resources, and thus contributing to environmental sustainability and the circular economy in the field of medical and dental prostheses.

### SUMMARY OF THE INVENTION

A first aspect of the present invention relates to a process for obtaining a dental prosthesis comprising a crown and tooth root. Said process preferably comprises the steps of:
a) directing undifferentiated cells towards an odontoblastic phenotype;
b) directing undifferentiated cells or keratinocytes towards an ameloblastic phenotype,
c) making a bioink available,
d) three-dimensional (3D) printing, preferably with 3D bioprinting, of the cells obtained in step a), the cells obtained in step b) and the bioink from step c), resulting in the dental prosthesis and
e) maintaining the dental prosthesis obtained in step d) in culture.

Preferably, the undifferentiated cells are mesenchymal cells, more preferably the mesenchymal cells are isolated cells from an individual's dental pulp.

Preferably, the keratinocytes are human-derived cells, deriving from a single adult or neonatal donor.

In one embodiment, in step a) the undifferentiated cells are maintained in a culture medium comprising a specific medium for mesenchymal cells based on the derivation.

In one embodiment, in step a) and step b) the cells are maintained in culture for at least 12 days, preferably for at least 14 days.

In a further embodiment, in step a) and step b) the cells are maintained in culture for at least 20 days, preferably for at least 25 days to achieve complete differentiation of the undifferentiated cells or keratinocytes into odontoblasts and/or ameloblasts.

Preferably, the bioink comprises a biodegradable, bioabsorbable or bioerodible material selected from the group consisting of chitosan, chitosan modified with acrylic groups, chitosan modified with methacrylic groups, chitosan modified with saccharide groups, alginate, alginate modified with acrylic groups, alginate modified with methacrylic groups, alginate modified with saccharide groups, cellulose and its derivatives such as methylcellulose, hydroxyethylcellulose, hydroxypropylmethylcellulose, polylactides, polyglycols, polycaprolactones polyanhydrides, polyamides, polyurethanes, polyesteramides, polyethers, polydioxanones, polyacetals, polyketals, polycarbonates, polyorthocarbonates, polyphosphazenes, polyhydroxybutyrates, polyhydroxyvalerates, polyalkylene oxalate, polyalkylene succinate, polymeric acid, polyamino acids, polymethylvinylether, chitin, collagen of all types, gelatine, gelatine modified with methacrylic groups, proteoglycans, chondroitin sulphate, silk proteins, keratan sulphate, dermatan sulphate, glycosaminoglycans and copolymers, polypropylene glycol alginate, polyglycol and polylactic acids, terpolymers and any combination thereof. Preferably, the bioink comprises at least one filler, preferably hydroxyapatite and/or nanohydroxyapatite and/or a bioglass. Preferably, the three-dimensional printing step d) comprises at least one sub-step d1) of preparing a bioink for three-dimensional printing, wherein the bioink comprises an amount of ameloblasts between 1 and 15 million/ml of ink and an amount of odontoblasts between 1 and 15 million/ml of ink. Preferably, the 3D printing step d) is a 3D bioprinting step. Furthermore, the dental prosthesis is preferably subjected to at least one step of decellularisation, preferably by thermal blasting.

A second aspect of the present invention regards a dental prosthesis obtained with the above-described process.

A third aspect of the present invention relates to a bioink suitable for three-dimensional printing (3D), preferably for 3D bioprinting, comprising at least one alginate, at least one gelatine and optionally at least one filler, wherein the at least one alginate is selected from: alginate modified with acrylic groups, alginate modified with methacrylic groups and alginate modified with saccharide groups and wherein the at least one filler is selected from: hydroxyapatite and/or nanohydroxyapatite and/or a bioglass. Preferably, the at least one alginate is alginate modified with methacrylic groups and wherein the at least one filler is hydroxyapatite.

### BRIEF DESCRIPTION OF THE FIGURES

FIG. 1 shows an analysis of the viability of MG63 cells embedded in the bioink according to the present invention by means of the Alamar test. The data are represented as a mean of the values±standard deviation (n=7);

FIG. 2 shows the results of the Live/Dead test on MG63 cells embedded in the bioink according to the present invention. The images were acquired with a fluorescence microscope with 4X magnification. The live cells are stained green, the non-viable ones red;

FIG. 3 shows an analysis of the viability of MG63 cells embedded in the bioink comprising hydroxyapatite according to the present invention by means of the Alamar test. The data are represented as the mean of the values±standard deviation (n=7);

FIG. 4 shows the results of a biocompatibility test (LDH test) on MG63 cells of a bioink comprising hydroxyapatite according to the present invention; and

FIG. 5 shows the results of a biocompatibility test (LDH test) carried out on 3T3 cells for the purpose of confirming the biocompatibility of a bioink without the presence of a filler.

### DETAILED DESCRIPTION OF THE INVENTION

A first aspect of the present invention relates to a process for obtaining a dental prosthesis comprising a crown and tooth root.

In one embodiment, said process comprises the steps of:
a) directing undifferentiated cells towards an odontoblastic phenotype;
b) directing undifferentiated cells or keratinocytes towards an ameloblastic phenotype,
c) making a bioink available,
d) three-dimensional (3D) printing, preferably with 3D bioprinting, of the cells obtained in step a), the cells obtained in step b) and the bioink from step c), resulting in the dental prosthesis, and
e) maintaining the dental prosthesis obtained in step d) in culture.

Step a) and step b) are in other words steps of committing undifferentiated cells or keratinocytes towards an odontoblastic and ameloblastic phenotype.

In one embodiment, step a) and step b) are steps of complete differentiation of undifferentiated cells or keratinocytes into odontoblasts and ameloblasts. Preferably, the cultures of ameloblasts and odontoblasts are obtained by differentiation of undifferentiated cells, more preferably by differentiation of mesenchymal cells or keratinocytes.

In one embodiment, the mesenchymal cells derive from a donor's tissue; preferably, the mesenchymal cells are isolated from dental pulp.

The term “isolated” means that the mesenchymal cells of the dental pulp are cells isolated from a natural source or a progeny thereof, for example derived from cell proliferation. The mesenchymal cells of the dental pulp used are preferably derived from the tissue of the dental pulp of a tooth or donor tissue. Preferably, the mesenchymal cells of the dental pulp are primary cells that have not been transformed or immortalised. In a further embodiment, the mesenchymal cells of the dental pulp are cells that have been transformed and/or immortalised.

In particular, the mesenchymal cells employed for the invention derive and are extracted from dental pulp. Preferably, the adult mesenchymal cells of dental pulp derive from non-embryonal tissue of the dental pulp of a donor tooth and are preferably autologously derived. The mesenchymal cells of the dental pulp are derived from the dental pulp tissue of any tooth or donor tissue that has been differentiated into dental pulp tissue. The mesenchymal cells of the dental pulp used in the method of the invention are preferably cells of human dental pulp. The cells are extracted from the molar teeth of a patient. In one embodiment, after extraction the tooth is placed in a buffer solution comprising at least one antibiotic, preferably a phosphate buffer (PBS) comprising penicillin/streptomycin.

In one embodiment, dental pulp taken from an individual is mechanically dissociated to obtain small parts of pulp and then rinsed, preferably, with a balanced saline solution supplemented with an enzymatic solution for digesting tooth bud tissues, and then the extraction of undifferentiated stem cells is performed.

Preferably, the enzymatic solution, also containing antibiotics, comprises at least one enzyme selected from the group made up of collagenase or dispase. More preferably, the enzymatic solution comprises collagenase and dispase. According to a preferred embodiment, the collagenase is type I and is present in a concentration ranging from about 5 mg/ 25 ml to about 100 mg/ 25 ml. Preferably, the dispase is type I and is present in a concentration ranging from about 3 mg/ 25 ml to about 100 mg/ 25 ml.

In one embodiment, the dissociation of the dental pulp is completed by a mechanical step, for example mechanical stirring, for a time of between 30 minutes and 120 minutes, at a temperature of between 30 and 40° C., preferably about 37° C. Preferably, at the end of the incubation period, the enzymatic solution is inhibited with a culture medium, for example a DMEM HG medium comprising foetal bovine serum (FBS) and the cells are recovered after centrifugation. Preferably, the cell pellet obtained from centrifugation is suspended in complete DMEM HG medium supplemented with 100 μM ascorbic acid or a specific medium for mesenchymal cells.

In one embodiment, the keratinocytes are obtained from a tissue of a donor; they are preferably obtained from a donor's gum tissue.

In one embodiment, the culture of ameloblasts and the culture of odontoblasts are maintained separately, that is, not in co-culture.

In one embodiment, in step a) and step b) the cells are maintained in culture for at least 12 days, more preferably for at least 14 days. In this period, the undifferentiated cells are directed towards an odontoblastic phenotype, in step a), and towards an ameloblastic phenotype in step b).

In one embodiment, the cells obtained in step a) and step b), i.e. cells directed towards an odontoblastic or ameloblastic phenotype, are mixed with the bioink from step c), printed with the 3D printer, in step d) and thereafter maintained in culture to achieve complete differentiation into odontoblasts and in ameloblasts.

In a further embodiment, in step a) and step b) the cells are maintained in culture for at least 20 days, more preferably for at least 25 days to achieve complete differentiation of the undifferentiated cells or keratinocytes into odontoblasts or ameloblasts. In other words, steps a) and b) are prolonged to achieve complete differentiation of the undifferentiated cells or keratinocytes in order then to mix them with the bioink and print them three-dimensionally.

In one embodiment, the culture of odontoblasts is obtained by means of techniques known to the person skilled in the art; preferably, mesenchymal cells of the dental pulp are isolated and cultured in a culture medium as described above.

Preferably, the mesenchymal cells are maintained under adherent conditions (2D) with a standard medium. In one embodiment, use is made of MEM (Minimum Essential Medium) or standard DMEM (Dulbecco's Modified Eagle Medium), or foetal bovine or calf serum (FBS or FCS); said serum is preferably present in a concentration of between 5% and 15% volume/volume (v/v), more preferably in a concentration of 10% FBS. According to another embodiment, the addition of serum can be gradually increased during every change of culture medium, starting from a concentration of about 5% until reaching a concentration of about 15%.

In one embodiment, the culture medium comprises at least one antibiotic and essential and/or non-essential amino acids and/or a calcium source.

Preferably, in order to obtain a culture of odontoblasts, the mesenchymal cells are maintained in culture in a medium comprising growth factors and/or differentiating factors selected from: ascorbic acid, β-glycerophosphate, and dexamethasone in concentrations of between 0.1 and 500 μM.

In one embodiment, the culture of ameloblasts is obtained by means of techniques known to the person skilled in the art; preferably, human-derived keratinocytes from an adult or neonatal donor are cultured in a culture medium. For example, the keratinocytes are obtained by separating the epidermis from the dermis using at least one enzyme or an enzymatic mixture. The keratinocytes are preferably separated from the collected tissue by mechanical and/or magnetic stirring, or by density gradient centrifugation or thanks to gravity-assisted cell sorting (GACS) based on a passive filtration of the suspension of keratinocytes through special nylon filters or with specific marker antibodies or by enzymatic digestion with the combined use of collagenase and dispase in a concentration of between 0.1 and 10 mg/mL, more preferably in a concentration of 2 mg/mL. The enzymatic digestion takes place for a time of 2 hours at a temperature of between 30 and 40° C., more preferably at 37° C. The tissue is then further digested with trypsin-EDTA at a concentration of between 0.01 and 1%, more preferably with 0.05% trypsin for 5 minutes at a temperature of between 30 and 40° C., more preferably at 37° C.

Preferably, the keratinocytes are maintained in a standard medium under adherent conditions above an already differentiated layer of mesenchymal cells, preferably of pulp differentiated into odontoblasts. In one embodiment, use is made of an MEM medium (Minimum Essential Medium) or DMEM (Dulbecco's Modified Eagle Medium) with foetal or calf bovine serum (FBS or FCS); said serum is preferably present in a concentration of between 5% and 15% volume/volume (v/v), more preferably in a concentration of 10% FBS. According to another embodiment, the addition of serum can be gradually increased during every change of culture medium, starting from a concentration of about 5% until reaching a concentration of about 15%.

In one embodiment, the culture medium comprises at least one antibiotic and essential and/or non-essential amino acids and/or a calcium source. Preferably, the calcium content is between 0.1 mM and 5 mM or a specific medium for keratinocytes with a calcium content between 0.1 mM and 5 mM.

Preferably, in order to obtain a culture of ameloblasts, the keratinocytes are maintained in culture in a medium comprising growth factors and/or differentiating factors and/or recombinant proteins preferably selected from: FGF-8, SHH, BMP 4 with a concentration ranging from 10 to 100 μg/mL.

In one embodiment, the bioink comprises a material of natural origin or of synthetic origin.

Preferably, the bioink comprises a biodegradable, bioabsorbable or bioerodible material selected from the group consisting of chitosan, chitosan modified with acrylic groups, chitosan modified with methacrylic groups, chitosan modified with saccharide groups, alginate, alginate modified with acrylic groups, alginate modified with methacrylic groups, alginate modified with saccharide groups, cellulose and its derivatives such as methylcellulose, hydroxyethylcellulose, hydroxypropylmethylcellulose, polylactides, polyglycols, polycaprolactones polyanhydrides, polyamides, polyurethanes, polyesteramides, polyethers, polydioxanones, polyacetals, polyketals, polycarbonates, polyorthocarbonates, polyphosphazenes, polyhydroxybutyrates, polyhydroxyvalerates, polyalkylene oxalate, polyalkylene succinate, polymeric acid, polyamino acids, polymethylvinylether, chitin, collagen of all types, gelatine, gelatine modified with methacrylic groups, proteoglycans, chondroitin sulphate, silk proteins, keratan sulphate, dermatan sulphate, glycosaminoglycans and copolymers, polypropylene glycol alginate, polyglycol and polylactic acids, terpolymers and any combination thereof.

In a preferred embodiment, the bioink is a chitosan-based or gelatine-based or alginate-based bioink. Preferably, the bioink further comprises at least one filler, preferably hydroxyapatite and/or nanohydroxyapatite and/or a bioglass.

In one embodiment, the three-dimensional printing step d) comprises at least one sub-step d1) of preparing a bioink for three-dimensional printing. Preferably, the bioink further comprises the ameloblasts and odontoblasts obtained in steps a) and b).

In a preferred embodiment of the invention, the bioink comprises an amount of ameloblasts between 1 and 15 million/ml, preferably about 10 million/ml, and an amount of odontoblasts between 1 and 15 million/ml, preferably about 10 million/ml.

In one embodiment, step d) comprises three-dimensional printing of the dental prosthesis, preferably bioprinting, more preferably using a 3D printer suitable for bioprinting.

Preferably, the printing of the dental prosthesis takes place after an image has been obtained of the oral cavity or a portion thereof, preferably the dental arches or a portion thereof of the individual in whom the dental prosthesis will subsequently be implanted.

In one embodiment, at least one image of the desired area is acquired, preferably of the oral cavity, with computed tomography (CT) or cone beam computed tomography (CBCT).

In a further embodiment, the image of the oral cavity is acquired thanks to a dental impression of the individual which is combined with the at least one acquired image.

Preferably, the acquired image is imported into implant treatment planning software.

Preferably, the dental prosthesis is designed to be in harmony and function with the surrounding teeth.

In one embodiment, the image obtained is converted into a three-dimensional image using software known to the person skilled in the art. Preferably, said three-dimensional image is imported into computer-aided design (CAD) software and the dental prosthesis is designed.

In one embodiment, the dental prosthesis designed via CAD is imported into 3D printing software known to the person skilled in the art. Said software is preferably “slicer” software. Preferably, said slicer software controls the print settings for the dental prosthesis, such as, for example, the print layer height, fill density, print speed, extrusion pressure, temperature of the heated bed, and volume of the dental prosthesis.

In one embodiment, after step d), in a step e), the dental prosthesis obtained is maintained in culture until allowing the formation of enamel and dentin in the dental prosthesis. Preferably, the dental prosthesis is maintained in culture under conditions known to the person skilled in the art, more preferably in an MEM medium (Minimum Essential Medium) or standard DMEM (Dulbecco's Modified Eagle Medium) with foetal bovine or calf serum (FBS or FCS); said serum is preferably present in a concentration of between 5% and 15% volume/volume (v/v), more preferably in a concentration of 10% FBS. According to another embodiment, the addition of serum can be gradually increased during every change of culture medium, starting from a concentration of about 5% until reaching a concentration of about 15%.

In one embodiment, the culture medium comprises at least one antibiotic and essential and/or non-essential amino acids and/or a calcium source.

Preferably, the dental prosthesis is maintained in culture for at least 5 days, more preferably for at least 10 days and at a temperature of between 30 and 40° C., preferably about 37° C.

At the end of step e) of culturing the dental prosthesis, said prosthesis is decellularised with techniques known to the person skilled in the art. Preferably, the dental prosthesis is decellularised by thermal blasting and/or by means of other treatments of the prosthesis in order to eliminate all the cells.

In a further embodiment, the process described above in detail is also suitable for the preparation of a bone graft for replacing a dental bone.

At the end of step e), the dental prosthesis is preferably subjected to at least one quality control step and at least one step of cleaning and sterilisation of the prosthesis itself.

The Applicant has surprisingly developed a process for preparing a dental prosthesis that has numerous advantages compared to the prostheses described and present on the market. First of all, the use of a biodegradable bioink makes it possible to considerably reduce the environmental impact of dental prostheses compared to the commercially available prostheses that use synthetic materials.

For example, the use of the prosthesis obtained with the above-described process makes it possible to considerably reduce the number of sittings a patient goes through for the implantation of the prosthesis, generally speaking from 10 sittings to 3 or 4 sittings with the prosthesis described. Furthermore, the prosthesis described perfectly replicates, from an aesthetic viewpoint, the shape and colour of the patient's natural teeth and there are no risks of rejection of the prosthesis or infections, and the risks of infiltration or lesions of the roots, nerves, or maxillary sinuses near the area of intervention are greatly reduced.

In addition, the implantation of the dental prosthesis can take place simultaneously with extraction of the tooth and is possible even in the case of infections, if not acute.

A second aspect of the present invention regards a dental prosthesis obtained with the process described above in detail.

A third aspect of the present invention regards a bioink for 3D printing. Preferably, said bioink is suitable for three-dimensional (3D) bioprinting. In one embodiment, the bioink comprises at least one alginate, at least one gelatine and optionally at least one filler.

Preferably, the at least one alginate is selected from: alginate modified with acrylic groups, alginate modified with methacrylic groups and alginate modified with saccharide groups; more preferably, it is alginate modified with methacrylic groups.

In one embodiment, the bioink comprises a weight/weight percentage of alginate between 0.05 and 5% w/w, preferably between 0.5 and 2% w/w.

In one embodiment, the bioink comprises a weight/weight percentage of gelatine between 5 and 25% w/w, preferably between 10 and 15% w/w.

Preferably, the at least one filler is selected from: preferably hydroxyapatite and/or nanohydroxyapatite and/or a bioglass; it is preferably hydroxyapatite.

In one embodiment, the bioink comprises a weight/weight percentage of filler between 0.5 and 5% w/w, preferably between 1 and 4% w/w.

In one embodiment, the bioink comprises a solvent, preferably water, in a percentage between 60 and 80% w/w, preferably between 5 and 75% w/w.

In one embodiment, the bioink comprises preservatives and/or pH stabilisers known to the skilled person.

In a preferred embodiment of the invention, the bioink comprises an amount of ameloblasts between 1 and 15 million/ml, and an amount of odontoblasts between 1 and 15 million/ml. Preferably, the bioink comprises cells directed towards an odontoblastic and/or ameloblastic phenotype, as described above in detail.

Preferably, the bioink is prepared with a method that envisages preparing a solution comprising the at least one alginate dissolved in a solvent and preparing a second solution comprising gelatine dissolved in at least one solvent. The two solutions are then mixed, preferably at a temperature of between 35 and 40° C.

Optionally, a third solution is prepared, comprising at least one filler as described above dissolved in a solvent and is mixed with the alginate solution and the gelatine solution.

A fourth aspect of the present invention regards a method for treating or preventing a dental condition or pathology associated with damage to or loss of at least one tooth.

In one embodiment, said method comprises at least one step of implanting a dental prosthesis as described above in detail to an individual who has a need therefor.

### EXAMPLE

### Preparation of the Bioink

### Solution A)

50 mg of Alginate methacrylate in 3.778 ml of water
0.05 ml Hepes 1M
0.825 ml of mannitol 0.9M

### Solution B) (Optional)

200 mg of hydroxyapatite in 0.825 ml of water
0.01 ml of Hepes 1M
0.165 ml of mannitol 0.9 M. The solution was sonicated before being added to the other solutions.

### Solution C)

2.5 grams of gelatine in 7.95 ml of water
0.1 ml of Hepes 1M
1.65 ml mannitol 0.9M

The solutions were mixed and placed under stirring at a temperature of about 40° C. for about 12 hours. Finally, the crosslinking elements were added.

The bioink obtained was maintained at a temperature of about 37° C. and then mixed with the cells. Once the ink was completed, it was transferred into a syringe suitable for bioprinting and irradiated with UV-visible light or visible light.

### Test on Printability and Cell Viability

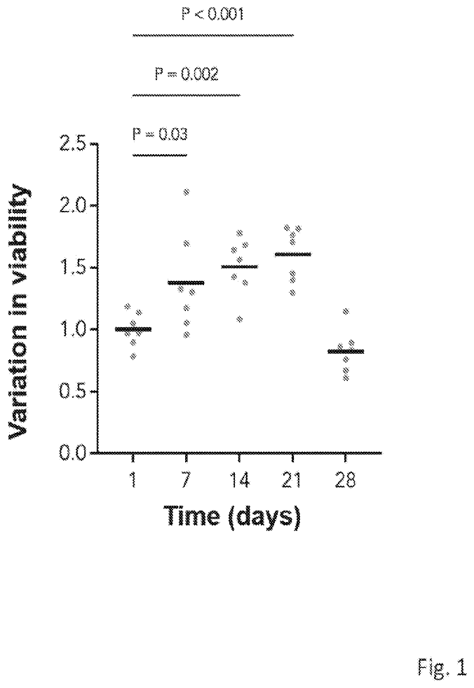

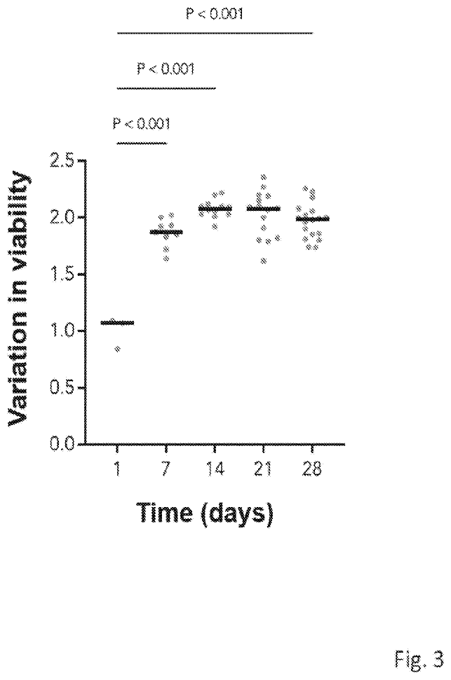

The viability of MG63 cells was evaluated with the Alamar test at different time intervals (after 1, 3, 7, 14, 21 and 28 days), both with a bioink without solution B, i.e. without hydroxyapatite, and a bioink comprising hydroxyapatite. The results obtained made it possible to confirm the biocompatibility of the bioink (FIG. 1 and FIG. 3)

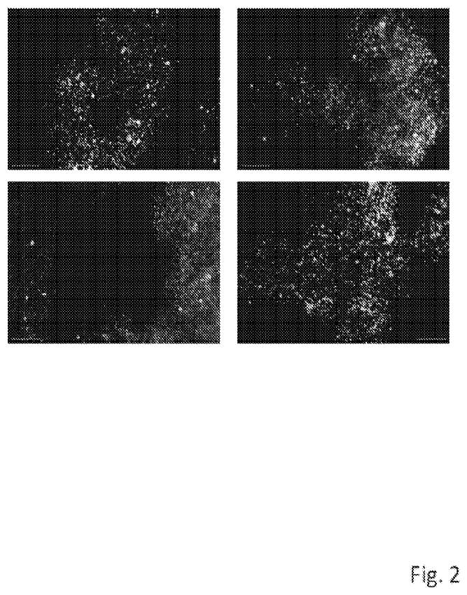

In order to confirm the cell viability results obtained by means of the Alamar test, a “live/dead assay” was performed. The latter is based on the use of a stain permeable to cell membranes which allows staining of live cells (green) and a nuclear stain impermeable to cell membranes of live cells, which thus allows staining of dead cells (red). The DNA of the cell is interspersed with the stain, which passes through damage present in the cell membrane of dead cells. (FIG. 2). The results obtained allowed a validation of the results obtained with the Alamar viability test.

Furthermore, in order to confirm the biocompatibility of a bioink comprising a filler and a bioink without a filler, preferably hydroxyapatite, a biocompatibility test (LDH Assay) was performed.

The biocompatibility of the bioink in question was evaluated following the guidelines of standard ISO 10993. The ink's biocompatibility was evaluated both by release (500 μL of bioink incubated at 37° C. in 3 mL of DMEM HG medium for 72 h and then administered to the cell population) and by contact (100 μL of bioink introduced in contact with the cell population in 500 μL of DMEM HG medium for 72 h).

As a positive control for toxicity, the cells were treated with 0.1% Triton-X 100 in DMEM HG. As a negative control for toxicity, by contrast, DMEM HG was used alone. The test was performed both 24 h and 72 h after the treatment. The toxicity data obtained were normalised on the basis of the toxicity values obtained from cell lysis. The biocompatibility of the bioink of the present invention was also compared with the biocompatibility of a bioink already available on the market and used for osteogenic differentiation. The biocompatibility of the bioink comprising a filler, preferably hydroxyapatite, was evaluated in a pilot cell line for osteogenic/odontogenic differentiation, namely MG63 cells. The biocompatibility of the bioink not comprising a filler was evaluated in a pilot cell line, 3T3.

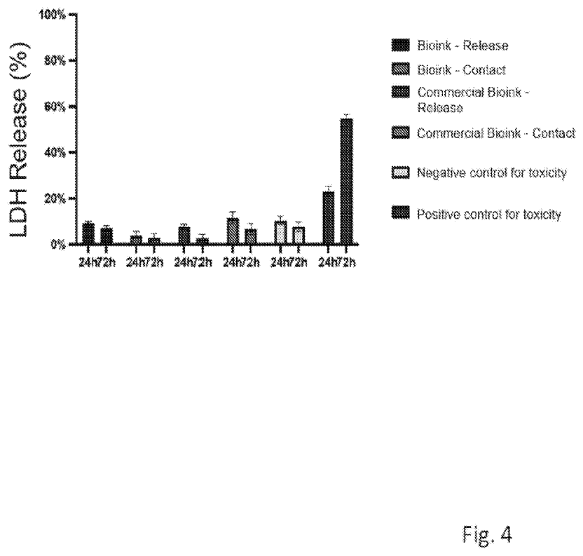

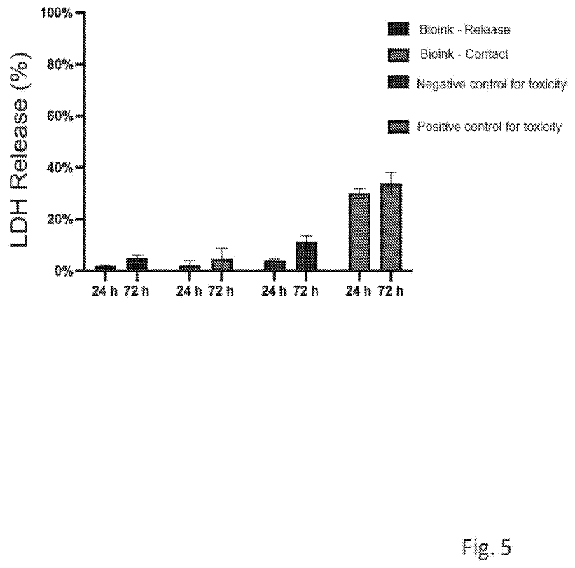

As is shown in FIGS. 4 and 5, all the bioink formulations show a toxicity (percentage of LDH release) that is drastically lower compared to the positive control for toxicity (Triton 0.1%). Furthermore, the bioink of the present invention shows a toxicity signal comparable to that of bioinks already available on the market and used for osteogenic differentiation and the release signal from cells not treated with material.

1. A process for obtaining a dental prosthesis comprising a crown and tooth root, this process comprising:
(a) directing undifferentiated cells towards an odontoblastic phenotype;
(b) directing undifferentiated cells or keratinocytes towards an ameloblastic phenotype,
(c) making a bioink available,
d) three-dimensional (3D) printing of the cells obtained in step a), the cells obtained in step b) and the bioink from step c), resulting in the dental prosthesis; and
e) maintaining the denture obtained in step d) in culture.

2. The process according to claim 1, wherein the undifferentiated cells are mesenchymal cells.

3. The process according to claim 2, wherein the mesenchymal cells are isolated cells from an individual's dental pulp and wherein the keratinocytes are isolated from gingival tissue.

4. The process according to claim 1, wherein in step a) and step b) the cells are maintained in culture for at least 12 days.

5. The process according to claim 1, wherein in step a) and step b) the cells are maintained in culture for at least 20 days to achieve complete differentiation of the undifferentiated cells or keratinocytes into odontoblasts and/or ameloblasts.

6. The process according to claim 1, wherein the bioink comprises a biodegradable, bioabsorbable or bioerodible material selected from the group consisting of chitosan, chitosan modified with acrylic groups, chitosan modified with methacrylic groups, chitosan modified with saccharide groups, alginate, alginate modified with acrylate groups, alginate modified with methacrylate groups, alginate modified with saccharide groups, cellulose and its derivatives comprising methylcellulose, hydroxyethylcellulose, hydroxypropylmethylcellulose, polylactides, polyglycols, polycaprolactones polyanhydrides, polyamides, polyurethanes, polyesteramides, polyethers, polydioxanones, polyacetals, polyketals, polycarbonates, polyorthocarbonates, polyphosphazenes, polyhydroxybutyrates, polyhydroxyvalerates, polyalkylene oxalate, polyalkylene succinate, polymeric acid, polyamino acids, polymethylvinylether, chitin, collagen of all types, gelatine, gelatine modified with methacrylic groups, proteoglycans, chondroitin sulphate, silk proteins, keratan sulphate, dermatan sulphate, glycosaminoglycans and copolymers, polypropylene glycol alginate, polyglycol and polylactic acids, terpolymers and any combination thereof.

7. The process according to claim 1, wherein the bioink comprises at least one filler.

8. The process according to claim 1, the three-dimensional printing step d) comprising at least one sub-step d1) of preparing a bioink for three-dimensional printing, wherein the bioink comprises an amount of ameloblasts between 1 and 15 million/ml and an amount of odontoblasts between 1 and 15 million/ml.

9. The process according to claim 1, wherein the dental prosthesis undergoes at least one step of de-cellularisation.

10. A dental prosthesis obtained by the process according to claim 1.

11. A bioink suitable for three-dimensional (3D) printing comprising at least one alginate, at least one gelatine and optionally at least one filler, wherein the at least one alginate is selected from: alginate modified with acrylic groups, alginate modified with methacrylic groups and alginate modified with saccharide groups, and wherein the at least one filler is selected from: hydroxyapatite, nanohydroxyapatite, a bioglass and combinations thereof.

12. The bioink according to claim 11, wherein the at least one alginate is alginate modified with methacrylic groups and wherein the at least one filler is hydroxyapatite.

13. The process according to claim 1, wherein step d) is 3D bioprinting.

14. The process according to claim 4 wherein in step a) and step b) the cells are maintained in culture for at least 14 days.

15. The process according to claim 5, wherein in step a) and step b) the cells are maintained in culture for at least 25 days.

16. The process according to claim 7, wherein said at least one filler is hydroxyapatite and/or nanohydroxyapatite and/or a bioglass.

17. The process according to claim 9, wherein the dental prosthesis undergoes at least one step of de-cellularisation by thermal blasting.
